(19) United States
(12) Patent Application Publication
(10) Pub. No.: US 2025/0352441 A1
(43) Pub. Date: Nov. 20, 2025

(54) DUAL PHASE ORAL CARE COMPOSITIONS

(71) Applicant: GURUNANDA, LLC, BUENA PARK, CA (US)

(72) Inventor: Puneet Nanda, Beverly Hills, CA (US)

(73) Assignee: GURUNANDA, LLC, BUENA PARK, CA (US)

(21) Appl. No.: 19/212,524

(22) Filed: May 19, 2025

Related U.S. Application Data
(60) Provisional application No. 63/649,783, filed on May 20, 2024.

(51) Int. Cl. A61K 8/22; A61K 8/19; A61K 8/34; A61K 8/60; A61K 8/92; A61Q 11/00

(52) U.S. Cl. CPC A61K 8/22; A61K 8/19; A61K 8/345; A61K 8/60; A61K 8/922; A61Q 11/00

(57) ABSTRACT

A dual phase oral care composition is disclosed herein comprising hydrogen peroxide and an activation composition.

### CROSS-REFERENCE TO RELATED APPLICATIONS

This application claims priority to, and the benefit of, U.S. Provisional Patent Application No. 63/649,783, filed May 20, 2024 and titled “DUAL PHASE ORAL CARE COMPOSITIONS,” which is incorporated by reference herein in its entirety for all purposes.

### TECHNICAL FIELD

The present disclosure relates generally to a dual phase composition for oral care comprising various constituents BACKGROUND

Most compositions for oral care, including mouth washes, dentifrices, and oral sprays, are aqueous. However, various compounds that may be beneficial in oral care may degrade at a rate such that they cannot be used in a product that has a commercially reasonable shelf-life stability.

### SUMMARY

In various embodiments, an aqueous oral care composition for oral care comprising: from about 0.001% to about 1% by weight sodium bicarbonate, from about 8% to about 15% by weight xylitol, from about 10% to about 17% by weight sorbitol 70%, from about 12% to about 17% by glycerin, and from about 60% to about 70% by weight water.

In various embodiments, an oral care composition system for oral care is provided comprising: a container comprising a first vessel and a second vessel, the first vessel at least partially containing a hydrogen peroxide solution and the second vessel at least partially containing an activation composition, wherein the hydrogen peroxide solution comprises: from 1% to 3% hydrogen peroxide by weight of the hydrogen peroxide solution, from 0.001% to 0.5% sodium benzoate by weight of the hydrogen peroxide solution, and wherein the activation composition comprises: from about 0.001% to about 1% by weight sodium bicarbonate; from about 8% to about 15% by weight xylitol; from about 10% to about 17% by weight sorbitol 70%; from about 12% to about 17% by glycerin; and from about 60% to about 70% by weight water.

### BRIEF DESCRIPTION OF THE DRAWING FIGURES

FIG. 1 illustrates a dual phase oral care composition system in a package according to various embodiments; and

FIG. 2 is a graph illustrating a method of making an oral care composition according to various embodiments.

### DETAILED DESCRIPTION

### Definitions

The following is a list of definitions for terms used herein. Unless defined otherwise, all technical and scientific terms used herein generally have the same meaning as commonly understood by one of ordinary skill in the art. In the event that there is a plurality of definitions for a term herein, those in this section prevail unless stated otherwise. Generally, the nomenclature used herein and the laboratory procedures in cytopathicity analysis, microbial analysis, organic, physical and inorganic chemistry, and dental clinical research are those well-known and commonly employed in the art.

As used herein, “about” will be understood by persons of ordinary skill in the art and will vary to some extent on the context in which it can be used. Generally, “about” encompasses a range of values that are plus/minus 10% of a reference value, unless specifically defined. For instance, “about 25%” encompasses values from 22.5% to 27.5%.

As used herein, “ambient conditions” means approximately room temperature (e.g., 20-35° C.) and relative humidity of approximately <70%.

As used herein, “a reasonable period of time” means the time, ranging from months to years, depending upon the application, a composition may be expected to maintain a safe and efficacious amount of its combined ingredients.

As used herein, “shelf-life stable” and “shelf-life stability” are used interchangeably and refer to the dual phase component composition being deemed consumer acceptable after a defined period of time after its production (under ambient conditions).

As used herein, “bioavailability” means to the absorption or penetration of the active agent(s) of the composition into the organic matter to which it is exposed and/or the absorption rate proportion of the dose of the composition that reaches the systemic circulation of the organic matter for which its use is intend. For example, when a composition is administered intravenously, its bioavailability is nearly 100%, while when the composition is administered topically, a fraction of the total composition reaches systemic circulation. The term “bioavailability” also refers to its availability for efficacy at the desired site and for efficacy that either intracellular, extracellular or within biofluids/biological fluids.

As used herein, “biocidal”, “bactericidal”, “fungicidal” or synonymous terms means the property of inactivating or killing microorganisms, such as bacteria, algae, yeast, and fungi. As used herein, “biocidal” also refers to the effect of a composition as a treatment for reduction of bacterial or fungal or microbial growth or overgrowth in fluids or biofilm which may be associated with alleviating a diseased condition or state.

As used herein, “anti-microbial” means the property of inactivating or killing microorganisms, such as viruses, bacteria, algae, yeast, and fungi. As used herein, “anti-microbial” also refers to the effect of a composition as a treatment for reduction of bacterial or fungal or ither microbial growth or overgrowth in fluids or biofilm which may be associated with alleviating a diseased condition or state.

As used herein, “biostatic”, “bacteriostatic”, fungistatic” or synonymous terms means the property of arresting the growth of microorganisms, such as bacteria, algae, yeast and fungi. As used herein, “biostatic” means to the effect of a composition in maintaining the polymicrobial mixture of a fluid or a biofilm, as in maintaining the oral ecology so that one or more organisms have not overgrown to enable inflection and disease. Compositions with biostatic attributes are useful in health maintenance, wellness and prevention of infection and disease.

As used herein, a “biofilm” means a biological aggregate that forms a layer on a surface, the aggregate comprising a community of microorganisms embedded in an extracellular matrix of polymers and/or other biocompounds such as glycoproteins. Typically, a biofilm comprises a diverse community of microorganisms, including bacteria (aerobic and anaerobic), algae, protozoa, yeast, and fungi. While monospecies biofilms also exist, biofilms in vivo become polymicrobial as they develop overtime creating oxygen-scare environments where anaerobic pathogens thrive and where the biofilm matrix protects the polymicrobial mixture within from antimicrobial treatment.

As used herein “a carrier” means those components of a composition that are capable of being commingled to provide required physical consistency and consumer goodness properties without interaction with other ingredients.

As used herein, “orally acceptable carrier” means a suitable vehicle or ingredient, which can be used to form and/or apply the present compositions to the oral cavity in a safe and effective manner.

As used herein, “compatible” means that the components of the composition are capable of being commingled without interaction in any manner which would substantially reduce the stability of the oral care composition, ingredients required for the efficacy, the carrier and excipients, and the consumer qualities of the composition.

As used herein, “consumer goodness qualities” include, but are not limited to, appearance, viscosity, taste, odor, abrasiveness, color, flavor, and moisturizing attributes of the compositions deemed desirable by consumers through consumer product testing or other such means. For example, it may be desirable that a tube of toothpaste produce a ribbon stripe of toothpaste on a toothbrush when squeezed and that the toothpaste composition is neither too firm to be squeezed easily from the tube nor too viscous so as not to hold or rest on the toothbrush ready or use.

As used herein, “dental plaque” means a polymicrobial biofilm that forms on the surface of teeth.

As used herein, “essentially free” means a composition which is comprised of very low levels, below detection levels of commonly used analytical methods, of a specific ingredient or compound or molecule.

As used herein, “vehicle” means an orally-acceptable dentifrice vehicle used to prepare a dentifrice composition comprising a water-phase, containing a humectant therein.

As used herein, “dentifrice” means paste, gel, powder, tablets, or liquid formulations, unless otherwise specified, that are used to clean the surfaces of the oral cavity.

As used herein, “teeth” refers to natural teeth as well as artificial teeth or dental prosthesis.

As used herein, “efficacious amount” means any amount of the agent that may result in a desired biocidal or biostatic or chemical or physiological effect, a desired cosmetic effect, and/or a desired therapeutic biological effect. In one example, an efficacious amount of an agent used for tooth whitening may be an amount that may result in whitening of a tooth with one or more treatments. In another example, an efficacious amount of an agent used for wound treatment is an amount that may result in a statistically significant improvement in wound healing.

As used herein, “film” means a layer of a material having two dimensions substantially larger than the third dimension. A film may be a liquid or a solid material. For some materials, a liquid film can be converted into a solid film by curing, for instance, by evaporation, heating, drying, cross-linking, adhering, adduct formation, and like phenomena.

As used herein, “hard tissue” means any toe and fingernail, hard keratinized tissue, hard tooth tissue, bone, tooth and the like, found in animals such as mammals.

As used herein, “essentially free of” means including unavoidable levels of a given material but no more.

As used herein, “substantially free of” means no more than 0.5% by weight of a given material.

As used herein, “irritating” and “irritation” refer to the property of causing a local inflammatory response, such as reddening, swelling, itching, burning, or blistering, by immediate, prolonged, or repeated contact. For example, inflammation of a non-oral mucosal or dermal tissue in a mammal can be an indication of irritation to that tissue. A composition may be deemed “substantially non-irritating” or “not substantially irritating,” if the composition is judged to be slightly or not irritating using any standard method for assessing dermal or mucosal irritation.

As used herein, “pharmaceutically acceptable” is set forth broadly and refers without limitation to those compounds, materials, compositions and/or dosage forms which are, within the scope of sound medical judgment suitable for contact with the tissues of and/or for consumption by human beings and animals without excessive toxicity, irritation, allergic response, or other problem complications commensurate with a reasonable risk/benefit ratio.

As used herein, the abbreviation “ppm” means parts per million by weight or volume as applicable.

As used herein, “overgrowth” refers to excessive concentrations of bacteria, algae, yeast, and/or fungi leading to inflammation, infection, pathogenesis and disease. Overgrowth may occur in biofilms and plaques containing polymicrobial mixtures of bacteria, algae, yeast, and/or fungi, such as those found in the biofilms associated with mucositis and with dental plaque. Overgrowths of pathogenic microbes within biofilms are known to increase significantly their resistance to treatment and increase the incidence of inflamed tissues, infection and disease.

As used herein, “prophylactic” means treatment administered to a subject who does not exhibit signs of a disease or exhibits early signs of the disease for the purpose of decreasing the risk of developing pathology associated with the disease.

As used herein, “range” means the area of variation between upper and lower limits on a particular scale. It is understood that any and all whole or partial integers between any ranges set forth herein are included herein.

As used herein, “safe and effective amount” and similar terms mean an amount of an ingredient in composition of sufficient dosage to positively modify the condition to be treated, but low enough to be safe for humans and animals to use without serious side effects (at a reasonable benefit/risk ratio), within the scope of sound medical/dental judgment. “Safe and effective” pertains not only to the dosage amount but also the dosage rate (rate of release) of an active compound or compounds.

As used herein, “stability” means the prevention of a reaction, reduction or degradation of components comprised in an oral care composition. An oral care composition may be “stable” if the components of the same are not reactive with each other for a reasonable period of time. For example, an oral care composition may be stable if it maintains consumer qualities for a period of 24 months at about 25° C. (ambient temperature) or 6 months at an accelerated temperature of 40°±2° C. and 75%±5% Relative Humidity (RH).

As used herein, “shelf-life” means the length of time compositions maintain the desired stability of the constituent compounds of the oral care composition and the consumer qualities of the composition.

As used herein, “therapeutic” means intended to be administered to a subject who exhibits signs of pathology for the purpose of diminishing or eliminating those signs.

As used herein, “topical composition” means a product which is not intentionally ingested or otherwise applied without recovery for purposes of systemic administration of therapeutic agents, but is retained in the anal, vaginal, aural, oral, nasal, ocular, or urogenital cavities or upon the skin or other outer surfaces of the body, or upon an area of affected soft tissue for a time sufficient to contact substantially all of the surfaces and/or tissues for purposes of administration and delivery of therapeutic agents.

As used herein, “wound” means a laceration, abrasion, puncture, burn, and/or other injury to any one or more soft and/or hard tissue. Exemplary tissues considered for such wound treatment include mucosal tissue and dermal tissue including epidermal tissue, dermal tissue, and subcutaneous tissue (also called hypodermis tissue). As used herein, a wound also encompasses a laceration, a puncture, and/or an avulsion of a fingernail or toenail. A wound can penetrate the tissue partially or completely. A wound can arise accidently or intentionally, e.g., a surgical wound.

As used herein, “dispersing agent” means a compound that improves the separation of particles and prevents settling or clumping of an ingredient(s) in a multicomponent composition.

As used herein, “emollient agent” means a compound that reduces the loss of water from a composition.

As used herein, “suspending or emulsifying agent” means a compound that achieves uniform dispersion of an ingredient(s) in a multicomponent composition.

As used herein, “fragrance” means a compound that provides a pleasing scent or order similar to perfume to a composition.

As used herein, “cooling agent” means a compound that provides a cooling, soothing, or pleasant feeling when a composition is topically applied to hard and soft tissues.

As used herein, “warming agent” means a compound that provide an olfactory sensation, especially warm sensation. Warming agents are often desired in various cosmetic preparations, such as shaving creams, hand lotions, body lotions, facial preparations, including masks, depilatories.

As used herein, “humectant” means a compound that preserves moisture in a composition. Some embodiments described herein include one or more compounds such as Glycerin, Sorbitol, Propylene Glycol, Propanediol, and Xylitol.

As used herein, “dual phase composition” means a composition wherein certain ingredients are contained in one part (e.g., container or vessel) and other ingredients are contained separately in a second part (e.g., container or vessel) at the time of manufacture and prior to use to prevent the reactivity of the certain components in each respective part. The bioavailability of dual phase compositions may be determined once the two phases are mixed at the time of use. A difference between single phase and dual phase compositions may include how shelf-life is determined. Because the two phases of a dual phase compositions are combined just prior to usage, the shelf-life stability of dual phase compositions is enhanced because components of each phase are held separately during manufacture and sale and then mixed only at or shortly prior to introduction into a body cavity, such as the oral cavity. In that regard, one or more chemical reactions may occur that improve the functionality of the combined oral composition.

As used herein, “thickener” means a compound that increases viscosity of a composition.

As used herein, “excipient” means a compound that provides physical and consumer goodness properties to a composition for its acceptance. Examples of such properties (but not limited to) are viscosity, appearance, flavor, color, thickness, sweetness, gel like structure, preservative, uniform suspension or combinations thereof.

As used herein, the term “abrasive agent” means a compound that helps to remove coating (or deposits) from hard or soft tissues, such as that on a tooth surface while brushing using a composition, such as a toothpaste.

As used herein, “desensitizing agent” means a compound that helps reduce or alleviate sensitivity and pain. For example, a desensitizing agent in a topical resin, varnish, toothpaste or mouthwash may occlude dentin tubules or may desensitize nerve fibers, blocking the neural transmission.

As used herein, “functional agent” means a compound that performs a health or aesthetic enhancing function. For example, functional agents may be. For example, a desensitizing agent in a topical resin, varnish, toothpaste or mouthwash may occlude dentin tubules or may desensitize nerve fibers, blocking the neural transmission.

All percentages and ratios used herein are by weight of a multi-component composition and not of the overall topical formulation that is delivered, unless otherwise specified. All measurements are made at room temperature i.e. 20-25° C., unless otherwise specified. Heat and humidity, under normal circumstances, may cause such a composition to degrade from liquid to gas, changing its weight and rendering common assay calculations inaccurate.

### Detailed Description of Embodiments

Disclosed herein, in various embodiments, are dual phase compositions for use in the oral cavity. Various compounds that may be used in oral care may degrade over days, weeks, or months, leading to a product that has a commercially unacceptable shelf life. In that regard, dual phase compositions allow such components to be stored under conditions that promote longer shelf life and then, when mixed with other components during or shortly prior to use, the compositions may be activated so as to be effective in the oral cavity. Oral care compositions serve a variety of purposes, including degrading compounds that produce malodor to mitigating microbial growth, promoting the remineralization of teeth, improving tooth microhardness, whitening teeth, among other benefits. Moreover, oral care compositions are able to deliver water soluble (i.e., hydrophilic) vitamins to tissues in the oral cavity.

In various embodiments, dual phase compositions comprise a first part and a second part. The first part and the second part are held in separate vessels, containers, or volumes for storage during manufacture and sale of the dual phase composition. During or shortly prior to use, the first part is mixed with the second part and introduced into a bodily cavity, though of course the first part may be mixed with the second part in a bodily cavity, such as the oral cavity.

In various embodiments, the first part comprises hydrogen peroxide. Hydrogen peroxide may be present in various concentrations. For example, hydrogen peroxide may be present in 0.1% to 5.0%, from 1% to 4%, and/or from 1%-3%, where the percentage is given on a mass of solute relative to volume of solution. In this manner, 3% hydrogen peroxide means 3 g of hydrogen peroxide in 100 mL of solvent (here, water, unless otherwise specified). Any concentration of hydrogen peroxide that is generally considered as safe for use in the oral cavity is contemplated herein. Hydrogen peroxide decomposes to gaseous oxygen and water over time. The presence of various other compounds with hydrogen peroxide may cause hydrogen peroxide to decompose too quickly to yield a product with a commercially reasonable shelf life.

In this manner, a first part may comprise hydrogen peroxide maintained a pH low enough to resist or delay decomposition. In various embodiments, the pH of the hydrogen peroxide solution is between 3 to 6, and/or between 3.5 and 4.5. Increasing the pH of the hydrogen peroxide solution may cause more rapid decomposition of the hydrogen peroxide.

In various embodiments, the second part comprises an activation composition comprising one or more of an accelerant, a functional agent, a water soluble vitamin, and an essential oil.

The accelerant, as used herein, may be any substance that accelerates or otherwise brings about the decomposition of hydrogen peroxide into oxygen and water, whether by acting as a reactant with the hydrogen peroxide, as a catalyst to improve reaction kinetics, and/or to raise pH to between 6 to 8. In various embodiments, the accelerant comprises one or more of sodium bicarbonate (i.e., baking soda), sodium carbonate, sodium sesquicarbonate, sodium hydroxide, certain enzymes such as catalase, peroxidase, any safe for oral use transition metal compounds that can act as a source of transition metal ions, such as zinc, manganese, chromium or other suitable transition metals.

While not desiring to be bound by theory, it is believed that sodium bicarbonate may serve a catalytic function in the decomposition of hydrogen peroxide, increasing decomposition kinetics and causing a rapid conversion of hydrogen peroxide into gaseous oxygen and water. Moreover, a reaction between hydrogen peroxide and sodium bicarbonate may occur that produces sodium percarbonate. Sodium percarbonate is an oxidizing agent. In this manner, it is believed that the mixture of sodium bicarbonate and hydrogen peroxide both raises the pH of the resultant solution to between 6 to 8 and/or between 6.5 and 7.5, and/or between 6.9 and 7.1. Further, it is believed that the mixture of sodium bicarbonate and hydrogen peroxide causes the release of gaseous oxygen and/or the evolution of sodium percarbonate, both of which are oxidizing agents. Oxidizing agents in the oral cavity may breakdown or otherwise degrade malodorous compounds in the oral cavity and/or may have an antimicrobial effect. Oxidizing agents may also act on surface enamel of teeth to remove or mitigate staining. In addition, gaseous oxygen may assist in removing debris from the oral cavity. A neutral or near neutral pH of the resultant mixture of the first part and the second part may reduce the potential for inflammation and/or irritation in the oral cavity. This is important as inflammation in the gums of the oral cavity is associate with various oral disease states.

The second part may further comprise a functional agent. The functional agent may be any material that is capable of supporting oral health and/or improving the function of the hydrogen peroxide and accelerant system. In various embodiments, xylitol is present in the second part. Xylitol may be present in the amount of 0.5% to 15%, 3% to 12%, and from 5% to 10%, where again percentage means mass of solute per volume of solution. Xylitol is known to promote saliva production, which can further help to neutralize the acidity in the oral cavity and alleviate irritation. Xylitol also has antibacterial properties that can reduce the risk of infections and reduce the sources of malodorous compounds. Moreover, xylitol helps to stabilize pH to a neutral or near neutral level in the oral cavity, protecting against tooth enamel damage that might otherwise occur if pH is lowered. In various embodiments, functional agents further include one or more of zinc gluconate (0.01-0.1% w/w, used a source of zinc ion which acts on volatile sulfur compounds to reduce oral malodor, Papain (0.1-0.5%, for natural teeth whitening), essential oils for multiple purposes (as described herein) such as antimicrobials, anti-inflammatory, saliva stimulation, desensitizer, analgesics, breath freshening etc., glycerin (15% w/w to act as humectant), sorbitol (10% w/w to act as humectant), calcium lactate (0.01-0.1% w/w to act as a source of calcium), any other suitable functional agent for taste masking, improving flavor profile and/or improving shelf life.

In various embodiments, the oral care composition may contain flavorings, anti-microbials, and/or excipients.

In various embodiments, the second part of the dual phase oral care composition comprises an essential oil. Essential oils such as botanical essential oils, for example, clove oil, oregano oil, tea tree oil (also known as melaleuca oil), thyme oil, peppermint oil, myrrh oil, Aegle marmelos oil, Ageratum oil, Cymbopogon nardus (citronella) oil, eucalyptus oil, cedarwood oil, lavender oil, cinnamon oil, lemongrass oil, lemon oil, orange oil, Cymbopogon martinii (palmarosa) oil, and patchouli oil and/or combinations of the same. Clove oil is rich in eugenol, which has various anti-microbial properties. Moreover, oregano oil and tea tree oil are known to have anti-microbial properties. Oregano oil contains various terpenes, phenols and terpenoids. For example, oregano oil may comprise carvacrol, thymol, and rosmarinic acid. Carvacrol is believed to have anti-bacterial properties while thymol is believed to have anti-fungal properties. Use of such essential oils tend to improve gum health, reduce plaque, and alleviate oral malodor. Oil and water phases will be mixed using emulsifying agent typically used in oral care products such as Polysorbates (including Polysorbate 20, Polysorbate 40, Polysorbate 60, and Polysorbate 80) and Sorbitan Esters (including Span 20, Span 40, Span 60, and Span 80), Polyethylene glycol (PEG) esters of fatty acids, Sodium lauryl sulfate (SLS), Sodium laureth sulfate (SLES), Sodium Lauroyl Sarcosinate, Cetylpyridinium chloride (CPC), Cocamidopropyl Beatine, Sodium Cocoyl Glutamate, Sodium Cocoyl Isethionate, Sodium Cocoyl Glycinate, Glyceryl Stearate, Lecithin, Sodium stearoyl lactylate, Xanthan gum, Carrageenan, Cellulose gum, Guar gum, Mono- and diglycerides

In various embodiments, the second part of the dual phase oral care composition comprises antimicrobial agents that include a diverse range of synthetic chlorinated substances, various polymers including peptides, and certain organosulfur compounds. Chlorinated substances include, but are not limited to, triclosan and triclocarban. Antimicrobial polymers include, but are not limited to, bacitracin, boceprevir, dalbavancin, daptomycin, enfuvirtide, oritavancin, teicoplanin, telaprevir, telavancin, vancomycin and guavanin-2. Antimicrobial organosulfur compounds include, but are not limited to, allicin, 3-vinyl-4H-1,2-dithiin, 2-vinyl-4H-1,3-dithiin, ajoene and diallyl disulfide.

In various embodiments, the anti-microbial agent may be present in an amount of from about 0.01% to about 10%, for example, from about 0.01%, 0.1%, 0.2%, 1%, 2%, 3%, 4%, 5%, 6%, 7%, 8%, or to about 10% by weight of the second part of the dual phase oral care composition. In various embodiments, the anti-microbial agent may be in an amount from about 0.15% to about 1.2%, from about 0.18% to about 1.0%, or from about 0.2% to about 0.3% by weight of the second part of the dual phase oral care composition. For the avoidance of doubt, the values recited here are of the percentage weight of the second part of the dual phase oral care composition relative to the total weight of the second part of the dual phase oral care composition.

In various embodiments, the oral care composition comprises a functional agent comprising an herbal extract. Herbal extracts comprise various phytochemicals that are extracted from herbs and/or other plants. In various embodiments, the oral care composition comprises an herbal extract extracted using supercritical carbon dioxide (CO₂). Supercritical carbon dioxide extraction comprises subjecting plant material to carbon dioxide at temperatures and pressures above the critical point of carbon dioxide to form a supercritical fluid. Supercritical carbon dioxide thus penetrates the plant matter and extracts various components from the plant matter, including various hydrophobic substances. The extract-laden supercritical carbon dioxide may be boiled in various stages, thus releasing the plant material components in a residual oil in each stage. In that regard, supercritical carbon dioxide may be tuned during various separation stages to produce fractions of herbal extracts grouped by molecular weight. Accordingly, herbal extracts may be fractionated into waxes, heavy oils/resins, and light oils. Stated another way, supercritical carbon dioxide extraction provides an effective way to remove targeted components from plant materials. In various embodiments, herbal extracts may be fractionated by molecular weight. However, in various embodiments, herbal extracts may contain all or substantially all types of substances present in the plant material from which it is extracted. Supercritical carbon dioxide extraction provides the ability to extract various substances from plant material without the use of volatile organic solvents such as hexane or ethanol.

In various embodiments, a herbal extract comprises ashwagandha extract, neem extract, cinnamon extract, neem extract, clove extract, roman chamomile extract, cardamom extract, tea tree oil extract, triphala extract, amla extract, sea buckthorn extract, licorice, echinacea extract, marigold extract, ginger extract, bacopa extract, hemp extract (CBD), schezwan extract, spilanthes extract, thyme extract, and sunflower oil extract, and turmeric (Curcuma longa) extract. In various embodiments, a herbal extract comprises supercritical carbon dioxide extracted ashwagandha (Withania somnifera), supercritical carbon dioxide extracted neem, supercritical carbon dioxide extracted cinnamon, supercritical carbon dioxide extracted neem, supercritical carbon dioxide extracted clove, supercritical carbon dioxide extracted roman chamomile, supercritical carbon dioxide extracted cardamom, supercritical carbon dioxide extracted tea tree, supercritical carbon dioxide extracted sunflower, and supercritical carbon dioxide extracted turmeric. Though known to one of skill in the art, supercritical carbon dioxide extracted hydrophobic herbal extracts are substantially free of residual organic solvents used in other extraction methods. Moreover, supercritical carbon dioxide extracted herbal extracts may be complete, meaning that such herbal extracts contain all or substantially all the types of phytochemicals in the raw plant material or supercritical carbon dioxide extracted herbal extracts may be fractionated, meaning that such herbal extracts contain a subset of the types of phytochemicals in the raw plant material. In this manner, essential oils, as used herein, may be extracted using conventional methods and capturing a wide spectrum of materials within the starting plant material whereas supercritical carbon dioxide extracted herbal extracts may be tuned by relative molecular weight to produce a selectable spectrum (e.g., narrow or wide) of materials.

Supercritical carbon dioxide extracted ashwagandha comprises alkaloids (isopelletierine, anaferine, cuseohygrine, anahygrine), steroidal lactones (withanolides, withaferins) and/or saponins. Supercritical carbon dioxide extracted ashwagandha is believed to have anti-inflammatory activity as well as other positive health benefits. Use in an oral care composition may help to reduce oral inflammation (e.g., gum inflammation) and anxiety that may lead to the grinding of teeth.

Supercritical carbon dioxide extracted turmeric comprises curcuminoids (curcumin). Supercritical carbon dioxide extracted turmeric is believed to have anti-inflammatory activity as well as other positive health benefits such as anti-anxiety health effects. Use in an oral care composition may help to reduce oral inflammation (e.g., gum inflammation).

In various embodiments, the supercritical carbon dioxide extracted herbal extract may be present in an amount of from about 0.01% to about 10%, for example, from about 0.01%, 0.1%, 0.2%, 1%, 2%, 3%, 4%, 5%, 6%, 7%, 8%, or to about 10% by weight of the second part of the dual phase oral care composition. In various embodiments, the herbal extract may be in an amount from about 0.15% to about 2%, from about 0.18% to about 1.5%, or from about 0.05% to about 1% by weight of the second part of the dual phase oral care composition. For the avoidance of doubt, the values recited here are of the percentage weight of the herbal extract relative to the total weight of the second part of the dual phase oral care composition. It is noted that various embodiments may have more than one the herbal extract. The values given herein relate to the sum of the herbal extracts in the second part of the dual phase oral care composition.

In various embodiments, the second part of the dual phase oral care composition comprises a flavoring agent.

In various embodiments, a flavoring agent comprises for example, spearmint oil, peppermint oil, cardamom oil, cinnamon oil, rose oil, jasmine oil, orange oil, lemon oil, vanilla oil and combinations thereof. In various embodiments, suitable flavoring agents can include, for example, flavors, such as, natural flavor oils, artificial flavors, and combinations thereof. Nonlimiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof. Other flavoring agents that may be included individually or in combination include, for example, anethole, menthol, menthone, L-menthyl acetate, eucalyptol, borneol, borneol acetate, camphor, 1,8-cineole, cinnamaldehyde, benzaldehyde, citral, thujone, eugenol, limonene, geraniol, citronellol, citronellal, pinene, linalool, thymol, carvone, caryophyllene, linalyl acetate, methyl salicylate, 3,3,5-trimethylcyclohexanol, methoxycyclohexanol, benzyl alcohol, anise alcohol, cinnamyl alcohol, β-phenyl ethyl alcohol (2-phenylethanol), cis-3-hexenol, musk xylol, isoeugenol, methyl eugenol, α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, hydroxycitronellal, benzaldehyde, methyl nonyl acetaldehyde, dodecanol, α-hexylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glycidate, methyl formate, isopropyl formate, linalyl formate, ethyl acetate, octyl acetate, methyl acetate, benzyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, β-phenylethyl acetate, trichloromethylphenyl carbinyl acetate, terpinyl acetate, vetiveryl acetate, and mixtures thereof.

In various embodiments, the flavoring agent may be present in an amount of from about 0.01% to about 10%, for example, from about 0.01%, 0.1%, 0.2%, 1%, 2%, 3%, 4%, 5%, 6%, 7%, 8%, or to about 10% by weight of the second part of the dual phase oral care composition. In various embodiments, the flavoring agent may be in an amount from about 0.15% to about 4%, from about 0.18% to about 3.0%, or from about 0.2% to about 2.5% by weight of the second part of the dual phase oral care composition. For the avoidance of doubt, the values recited here are of the percentage weight of flavoring agent relative to the total weight of the second part of the dual phase oral care composition. It is noted that various embodiments may have more than one flavoring agent. The values given herein relate to the sum of flavoring agents in the second part of the dual phase oral care composition.

In various embodiments, the second part of the dual phase oral care composition may contain one or more enzymes. Enzymes reduce the activation energy of certain reactions, acting as a catalyst. In various embodiments, the second part of the dual phase oral care composition comprises one or more of glucose oxidase, bromelain, catalase, or papain. In various embodiments, the second part of the dual phase oral care composition may comprise an oxidoreductase enzyme. For example, the second part of the dual phase oral care composition may comprise a peroxidase such as catalase. Catalase breaks down hydrogen peroxide (H₂O₂). While not desiring to be bound by theory, catalase contains a heme group (an iron-containing cofactor) at its active site. The iron atom in the heme group temporarily binds to one molecule of hydrogen peroxide, oxidizing it to form water and an oxygen atom. This oxygen atom then reacts with a second hydrogen peroxide molecule, reducing it to water and releasing oxygen gas (O₂). In the context of the second part of the dual phase oral care composition, when the catalase is mixed with the first part of the dual phase oral care composition that contains hydrogen peroxide, the catalase breaks down the hydrogen peroxide to release oxygen gas in the oral cavity. This rapid evolution of oxygen gas in the oral cavity aides in tooth stain removal as well as the oxidation of compounds that contribute to malodor, tooth decay, as well as contribute to anti-microbial effects.

In various embodiments, the second part of the dual phase oral care composition comprises a medicated agent. A medicated agent comprises any medication that is suitable for use in medical treatment. In various embodiments, the medicated agent is intended to treat a disease state and/or provide relief from symptoms of a disease state.

In various embodiments, the medicated agent comprises at least one of menthol, eugenol, camphor, capsicum, thymol, eucalyptol, alcohol, methylsalicylate, sodium fluoride, stannous fluoride, sodium monolfuorophosphate, strontium chloride, potassium nitrate, cetylpyridinium chloride, hydrogen peroxide, carbamide peroxide, sodium citrate, alum, benzocaine, benzyl alcohol, elm bark, glycerin, hexylresorcinol, pectin, phenol, phenolate sodium, salicyl alcohol, sodium bicarbonate, sodium perborate, zinc chloride, chlorophyllin, citric acid, beechwood, cresol, horehound, lidocaine, myrrh oil, sodium borate, sodium citrate, and/or tolu balsam.

In various embodiments, the medicated agent may be present in an amount of from about 0.01% to about 10%, for example, from about 0.01%, 0.1%, 0.2%, 1%, 2%, 3%, 4%, 5%, 6%, 7%, 8%, or to about 20% by weight of the second part of the dual phase oral care composition. In various embodiments, the medicated agent may be in an amount from about 0.005% to about 4%, from about 0.01% to about 3.0%, or from about 0.015% to about 2.5% by weight of the second part of the dual phase oral care composition. For the avoidance of doubt, the values recited here are of the percentage weight of the medicated agent relative to the total weight of the oral care composition. It is noted that various embodiments may have more than one medicated agent relative. The values given herein relate to the sum of medicated agents in the second part of the dual phase oral care composition.

In various embodiments, the oral care composition comprises a salivary stimulating agent. In various embodiments, a salivary stimulating agent comprises a material that is capable of triggering a salivary response in mammals. In various embodiments, salivary stimulating agents include, for example, cardamom oil, lavender oil, eucalyptus oil, fennel oil, schezwan oil (e.g., a light vegetable oil such as canola oil infused with chili peppers), spilanthes oil, menthol oil, and/or combinations of the same.

In various embodiments, the salivary stimulating agent may be present in an amount of from about 0.01% to about 10%, for example, from about 0.001%, 0.01%, 0.02%, 1%, 2%, 3%, 4%, 5%, 6%, 7%, 8%, or to about 10% by weight or volume of the second part of the dual phase oral care composition. In various embodiments, the flavoring agent may be in an amount from about 0.005% to about 0.04%, from about 0.01% to about 0.1%, or from about 0.015% to about 0.5%.

For the avoidance of doubt, the values recited here are of the percentage weight of salivary stimulating agent relative to the total weight of the second part of the dual phase oral care composition. It is noted that various embodiments may have more than one salivary stimulating agent. The values given herein relate to the sum of salivary stimulating agent in the second part of the dual phase oral care composition.

In various embodiments, the oral care composition comprises a hydrophilic vitamin. A hydrophilic vitamin may refer to a vitamin that is soluble in water. Hydrophilic vitamins include vitamin C, Vitamin B3 (Niacin), Vitamin B6 (Pyridoxine), Vitamin B12 (Cobalamin), Folate (Vitamin B9), Vitamin B2 (Riboflavin). In various embodiments, the oral care composition comprises a hydrophobic vitamin such as vitamin K, such as vitamin K2. Vitamin K2 facilitates calcium utilization by helping direct calcium to locations in the body where calcium utilization is important, such as teeth and bones, promoting better oral and skeletal health.

In various embodiments, the hydrophilic vitamin may be present in an amount of from about 0.01% to about 10%, for example, from about 0.01%, 0.1%, 0.2%, 1%, 2%, 3%, 4%, 5%, 6%, 7%, 8%, or to about 10% by weight of the second part of the dual phase oral care composition. In various embodiments, the hydrophilic vitamin may be in an amount from about 0.005% to about 1.2%, from about 0.18% to about 1.0%, or from about 0.2% to about 0.7% by weight of the second part of the dual phase oral care composition. For the avoidance of doubt, the values recited here are of the percentage weight of hydrophilic vitamin relative to the total weight of the second part of the dual phase oral care composition. It is noted that various embodiments may have more than hydrophilic vitamin. The values given herein relate to the sum of hydrophilic vitamins in the oral care composition.

In various embodiments, the second part of the dual phase oral care composition comprises a coenzyme. In particular, in various embodiments, second part of the dual phase oral care composition comprises a coenzyme. In various embodiments, the second part of the dual phase oral care composition comprises coenzyme Q10 (CoQ10), coenzyme A, coenzyme F420, coenzyme B, and coenzyme M, among others. In various embodiments, CoQ10 is included for a variety of benefits. CoQ10 has antioxidant properties which may improve the health of the gums, teeth, and other tissues of the oral cavity.

In various embodiments, the coenzyme may be present in an amount of from about 0.01% to about 10%, for example, from about 0.01%, 0.1%, 0.2%, 1%, 2%, 3%, 4%, 5%, 6%, 7%, 8%, or to about 10% by weight of the second part of the dual phase oral care composition. In various embodiments, the coenzyme may be in an amount from about 0.05% to about 1.2%, from about 0.1% to about 1.0%, or from about 0.2% to about 0.7% by weight of the second part of the dual phase oral care composition. For the avoidance of doubt, the values recited here are of the percentage weight of coenzyme relative to the total weight of the second part of the dual phase oral care composition. It is noted that various embodiments may have more than coenzyme. The values given herein relate to the sum of coenzyme in the second part of the dual phase oral care composition.

In various embodiments, the oral care composition comprises a sweetening agent. Suitable sweetening agents may be soluble in water. In various embodiments, the sweetening agent comprises sucrose, aspartame, acesulfame, stevia, saccharin; saccharin salts, especially sodium saccharin; sucralose, sodium cyclamate, and mixtures thereof. In various embodiments, sweetening agents that are polyhydroxy alcohols such as xylitol, mannitol, and sorbitol. In various embodiments, the oral care composition is essentially free of polyhydroxy sweeteners such as xylitol, mannitol, and sorbitol. In various embodiments, a sweetening agent comprises sucrose, stevia, allulose, sucralose, acesulfame, aspartame, cyclamate, or saccharin, and combinations thereof. In some embodiments, the sweetener may be in an amount of from about 0.01% to about 0.5%, for example, from about 0.01%, 0.02%, 0.05%, 0.1%, 0.2%, 0.3%, 0.4%, or to about 5.0% by weight or volume of the second part of the dual phase oral care composition. In various embodiments, the sweetener may be in an amount from about 0.05% to about 0.5%, from about 0.1% to about 0.2%, from about 0.01% to about 0.5%, or from 0.01% to about 0.2%.

In various embodiments, the oral care composition comprises a carrier. In various embodiments, suitable carrier(s) comprise those that satisfy various considerations based on compatibility with the other ingredients required for the efficacy, consumer qualities, cost, and contribution to shelf stability. In various embodiments, the selected carrier does not substantially reduce either the stability of the oral care composition or its efficacy. Examples of suitable carriers variously include gelling agents, whitening agents, flavoring systems, coloring agents, abrasive agents, foaming agents, desensitizing agents, dispersants, humectants, analgesic and anesthetic agents, anti-inflammatory agents, anti-malodor agents, anti-microbial agents, anti-plaque agents, anti-viral agents, biofilm disrupting, dissipating or inhibiting agents, cellular redox modifiers, antioxidants, cytokine receptor antagonists, dental anti-calculus agents, fluoride ion sources, hormones, metalloproteinase inhibitors, enzymes, immune-stimulatory agents, lipopolysaccharide complexing agents, tissue growth factors, vitamins and minerals, water, and mixtures thereof.

In various embodiments, the oral care composition may further comprise one or more additional functional agents. In various embodiments, an additional active ingredient may include one or more of the following additional ingredients: fluoride ion sources, anti-microbial agents, analgesic compounds, anti-inflammatory agents, anti-malodor agents, anti-plaque agents, anti-viral agent, biofilm disrupting, dissipation or inhibiting agents, hormones, enzymes, metalloproteinase inhibitors, immune-stimulatory agents, and numbing agents. In further embodiments, the oral care composition comprises one or more excipients including any of abrasives, humectants, thickeners, sweeteners, moisturizers, flavors, colors, fillers, and extenders. In various embodiments, use of the oral care composition disclosed herein may relieve toothache, decrease oversensitivity in the gums, freshen breath (reduce malodor), and taste pleasant.

In various aspects, the oral care composition comprises a pharmaceutically acceptable carrier and/or excipients. Pharmaceutically-acceptable carriers include one or more compatible solid or liquid materials, including diluents or encapsulating substances, which are suitable for topical administration to the human or animal body and provide physical action or consumer-goodness characteristics acceptable to the user. The pharmaceutical carriers and/or excipients may be combined into the oral care composition without interaction in any manner that would reduce the compounds therein, the consumer goodness qualities, the safety and effectiveness of the composition in treating or preventing anal, aural, vaginal, oral, nasal, ocular, urogenital, foot, and skin disorders, or diseases of the skin or foot and the inflammation and infection of tissues therein. The choice of a pharmaceutically acceptable carrier and/or excipient may be determined by the way the composition is to be introduced into the anal, vaginal, aural, oral, nasal, ocular, or urogenital cavity, or to be applied topically in foot care and skin care. The pharmaceutically acceptable carrier and/or excipient may depend on secondary considerations such as, but not limited to, consumer goodness qualities, costs and shelf-life stability.

In various embodiments, the pharmaceutically acceptable carrier and/or excipients may be in an amount of from about 0.01% to about 30%, for example, from about 0.01%, 0.1%, 1%, 2%, 3%, 4%, 5%, 6%, 7%, 8%, 9% or to about 10% by weight or volume of the second part of the dual phase oral care composition. In other embodiments, the pharmaceutically acceptable carrier may be in an amount from about 0.01% to about 60%, from about 0.01% to about 30%, or from about 0.01% to about 20%. For the avoidance of doubt, the values recited here are of the percentage weight of pharmaceutically acceptable carrier relative to the total weight of the second part of the dual phase oral care composition. It is noted that various embodiments may have more than one pharmaceutically acceptable carrier. The values given herein relate to the sum of pharmaceutically acceptable carrier in the second part of the dual phase oral care composition. In various embodiments, sea salt may be used as a pH adjuster/flavor enhancer, calcium lactate may be used as source of calcium, zinc gluconate may be used as a source of zinc ion (which neutralizes malodors for example, volatile sulfur compounds), aloe vera may be used as a soothing/moisturizing agent, sorbitol may be used as a humectant, PEG-40 may be used as an emulsifier, papain may be used as a whitening agent, sodium laurel sarcosinate may be used as an emulsifier, foaming agent and cleansing agent, jasmine oil maybe used a flavor enhancer and menthol may be used as a breath freshener.

In aspects, the second part of the dual phase oral care composition comprises a coloring agent. Preferably, the consumer goodness qualities of the oral care composition are not degraded by the coloring agent. Coloring enables the consumer to more readily ascertain usage and dosage. Certain colors of the composition may be deemed undesirable for certain anal, vaginal, aural, ocular, oral or urogenital applications. In various embodiments, no coloring agent is present. In various embodiments, a coloring agent includes, FD&C Blue No. 1 or titanium dioxide. In various embodiments, the coloring agent may be in an amount of from about 0.01% to about 10%, for example, from about 0.01%, 0.1%, 1%, 2%, 3%, 4%, or to about 5% by weight or volume of the second part of the dual phase oral care composition. In further embodiments, the coloring agent may be in an amount from about 0.0001% to about 5.0%, from about 1% to about 10%, or from about 0.01% to about 2%, or from 0.0001% to about 0.5%.

In various embodiments, an second part of the dual phase oral care composition further comprises one or more humectants. Suitable humectants include those that include at least one of the following: serves to keep pastes and gels and suspensions from hardening or losing their consumer goodness qualities when exposed to air, to add to the compositions a moist feel to the consumer goodness qualities and, for particular humectants orally applied, to impart desirable sweetness of flavor, such as toothpaste compositions. In various embodiments, the humectant comprises polyhydroxy alcohols, including arabitol, erythritol, glycerol, maltitol, mannitol, sorbitol, and/or xylitol. Polyhydroxy alcohols are commonly accepted excipients and most belong to the Generally Recognized as Safe (GRAS) category for pharmaceutical, cosmetic, and food products. Other compounds which provide moist texture for suitable formulations may also be used such as propylene glycol, propanediol etc. In various embodiments, sorbitol may be a humectant comprised in the second part of the dual phase oral care composition.

In various embodiments, the humectant may be present in an amount of from about 0.001% to about 70%, for example, from about 0.001%, 0.01%, 1%, 2%, 5%, 10%, 20%, 30%, 40%, 50%, or to about 70% by weight or volume of the second part of the dual phase oral care composition. In further embodiments, the humectant may be in an amount from about 1% to about 15%, from about 15% to about 55%, or from about 25% to about 55%.

In various embodiments, the oral care composition comprises a fluoride ion source. In various embodiments, no fluoride ion source is present. In various embodiments, the second part of the dual phase oral care composition includes free fluoride ions or covalently bound fluorine in a form that may be hydrolyzed by oral enzymes to yield free fluoride ions. Free fluoride ions comprise sodium fluoride, silver diamine fluoride, stannous fluoride, or indium fluoride. Covalently bound fluorine, which can be enzymatically hydrolyzed to yield free fluoride, may be provided by sodium monofluorophosphate. In various embodiments, sodium fluoride may be comprised in the second part of the dual phase oral care composition as the source of free fluoride ions.

In various embodiments, second part of the dual phase oral care composition further comprises a source of fluoride ion providing fluoride ions from about 0 ppm to about 5000 ppm, or from about 50 ppm to about 3500 ppm, from about 500 ppm to about 3500 ppm. In various embodiments, the fluoride ion source may be in an amount of from about 0% to about 2.0%, for example, from about 0.01%, 0.1%, 0.15%, 0.2%, 0.3%, 0.4%, 0.5%, 0.6%, 0.7%, 0.8%, or to about 2.0% by weight or volume of the second part of the dual phase oral care composition. In further embodiments, the fluoride ion source may be in an amount from about 0.0% to about 0.03%, from about 0.0% to about 0.7%, from about 0.1% to about 0.8%, from about 0.01% to about 0.07%, or from about 0.0% to about 0.8%. A composition is referred as fluoride-free when the source of fluoride ion source is 0% or when the composition is essentially free of fluoride as described herein.

In aspects, second part of the dual phase oral care composition further comprises a thickening or binding agent. The thickening or binding agent may provide desired consumer goodness qualities appropriate to the oral care composition, such as the desirable consistency or viscosity of the composition, to provide desirable dosage and to adhere to hard or soft tissues in a topical application. Examples of thickening or binding agents include carboxyvinyl polymers, seaweed derivatives such as carrageenan, hydroxyethyl cellulose, laponite, powdered polyethylene, and salts of cellulose ethers such as sodium carboxymethylcellulose and sodium carboxymethyl hydroxyethyl cellulose. Natural gums such as gum karaya, guar gum, xanthan gum, gum arabic, and gum tragacanth can also be used. Colloidal magnesium aluminum silicate or finely divided silica may be used as part of the thickening or binding agent to further improves texture. Higher concentrations of thickening agents can be used for chewing gums, lozenges (including breath mints), sachets, non-abrasive gels and gels intended for use in wound-healing, vaginal or oral disease. In various embodiments, for example, in various mouthwash or rinse formulations of the presently disclosed oral care compositions comprise no thickening or binding agents. Indeed, various mouthwash or rinse formulations of the presently disclosed oral care compositions have relatively low viscosities.

In various embodiments, the thickening or binding agent may be present in an amount of from about 0% to about 15%, for example, from about 0.01%, 0.1%, 1%, 2%, 3%, 4%, 5%, 6%, 7%, 8%, or to about 15% by weight or volume of the second part of the dual phase oral care composition. In various embodiments, the thickening or binding agent may be in an amount from about 0.1% to about 15%, from about 2.0% to about 10%, from about 4% to about 8%, from about 1.0% to about 4.0%, or from about 5.0% to about 7.0%.

In various embodiments, the oral care composition further comprises a desensitizing agent. The desensitizing agent may be provided for temporary relief from pain to hard or soft tissues. In various embodiments, the desensitizing agent comprises compounds such as strontium chloride, strontium acetate, arginine, hydroxyapatite, nano-hydroxyapatite (nano-HAp), calcium sodium phosphosilicate, potassium chloride or potassium nitrate. In various embodiments, the compositions may be essentially free of compounds, such as sodium lauryl sulfate, that irritate sensitive body cavities such as anal, vaginal, nasal, ocular, oral, and urogenital. Examples of sensitivities and resultant diseases oral cavity include canker sores, oral mucositis, and dry mouth.

In various embodiments, the oral care composition further comprises a preservative. In embodiments, the preservative comprises a methyl paraben, propyl paraben, disodium EDTA, benzyl alcohol, benzoic acid, or sodium benzoate, potassium sorbate, sorbic acid, ethanol, chlorohexidine, cetylpyridinium chloride, cetylpyridinium chloride, benzalkonium chloride, tea tree oil, herbal extracts such as citrus extracts, grapeseed extracts, neem extracts and other suitable preservatives for oral care application. In various embodiments, the preservative may be present in an amount of from about 0% to about 2%, for example, 0.01%, 0.1%, 1%, or 2% by weight or volume of the oral care composition. In further embodiments, the surfactant may be in an amount from about 0.1% to about 0.15%, from about 0.2% to about 1%, from about 0.01% to 0.5%, or from about 0.4% to about 0.8%.

In various embodiments, the dual phase oral care composition may be specifically formulated for use in humans and animals, for example in the form of rinses, gels, pastes, creams, washes, sprays, lozenges, therapeutic floss, tape, patches, compresses, or strips, for use in skin care, oral care, urogenital care, foot care, wound healing and as a solution used in irrigation devices for use in the oral and other body cavities.

In various embodiments, the oral care composition is suitable for a variety of indications, including treatment and prevention of oral or vaginal malodor, as well as ocular, anal, nasal and skin care and other topical uses. Suitable topical indications include anal, aural, oral, nasal, ocular, urogenital, foot-care and skin-care conditions and diseases. The oral care composition may be suitable for select indications, including antimicrobial, antiseptic, antioxidant, bactericidal and bacteriostatic, biofilm penetration, biofilm dissipation and reduction, coagulant, deodorant, desensitizing, disinfectant, fungicidal and fungistatic, herbicidal, tissue damage reduction, bleaching, stain removal, and tooth whitening. Compositions herein are suitable for use in a variety of forms, including rinses, gels, pastes, creams, washes, sprays, lozenges, floss, tape, patches, bandages, compresses, wraps, and strips.

### Exemplary Composition I: Dual Phase Oral Care Composition

The first part of various dual phase oral care composition may comprise water from about 90% to about 99.99% by weight of the total first part of the dual phase oral care composition. The first part of various dual phase oral care composition may further comprise sodium benzoate from 0.1% to 1% by weight of the total oral care composition, citric acid from 0.0001% to 1% by weight of the total oral care composition, hydrogen peroxide from 0.1% to 6% by weight of the total oral care composition.

The second part of various dual phase oral care composition may comprise water from about 50% to about 99.99% by weight of the total second part of the dual phase oral care composition. The second part of various dual phase oral care composition may further comprise sodium benzoate from 0.001% to 0.5% by weight of the second part of the dual phase composition, xylitol from 0.001% to 20% by weight of the total oral care composition, sea salt (sodium chloride) (e.g., celtic sea salt) from 0.0001% to 2% by weight of the second part of the dual phase composition, calcium lactate from 0.001% to 1% by weight of the second part of the dual phase composition, zinc gluconate from 0.0001% to 1% by weight of the second part of the dual phase composition, stevia from 0.001% to 0.5% by weight of the second part of the dual phase composition, sodium bicarbonate from 0.001% to 1% by weight of the second part of the dual phase composition, aloe vera from 0.001% to 1% by weight of the second part of the dual phase composition, sorbitol 70% 0.1% to 20% by weight of the second part of the dual phase composition, glycerin from 0.1% to 20% by weight of the second part of the dual phase composition, PEG-40 hydrogenated castor oil from 0.1% to 2% by weight of the second part of the dual phase composition, papain from 0.001% to 1% by weight of the second part of the dual phase composition, sodium lauroyl sarcosinate from 0.001% to 5% by weight of the second part of the dual phase composition, spearmint oil from 0.001% to 1% by weight of the second part of the dual phase composition, clove oil from 0.001% to 1% by weight of the second part of the dual phase composition, tea tree oil from 0.001% to 1% by weight of the second part of the dual phase composition, peppermint oil from 0.001% to 1% by weight of the second part of the dual phase composition, cardamom oil from 0.001% to 1% by weight of the second part of the dual phase composition, oregano oil from 0.001% to 1% by weight of the second part of the dual phase composition, fennel oil from 0.001% to 1% by weight of the second part of the dual phase composition, jasmine oil from 0.0001% to 1% by weight of the second part of the dual phase composition, vitamin E from 0.001% to 1% by weight of the second part of the dual phase composition, vitamin D from 0.00001% to 1% by weight of the second part of the dual phase composition, vitamin K2 from 0.00001% to 1.0% by weight of the second part of the dual phase composition, catalase from 0.01% to 1.0% by weight of the second part of the dual phase composition, and menthol from 0.0001% to 1% by weight of the second part of the dual phase composition.

In various embodiments, the first part of various dual phase oral care composition may comprise water from about 96% to about 99% by weight of the total first part of the dual phase oral care composition. The first part of various dual phase oral care composition may further comprise sodium benzoate from 0.3% to 0.7% by weight of the total oral care composition, citric acid from 0.0001% to 0.5% by weight of the total oral care composition, hydrogen peroxide from 1% to 3% by weight of the total oral care composition.

In various embodiments, the second part of various dual phase oral care composition may comprise water from about 60% to about 70% by weight of the total second part of the dual phase oral care composition. The second part of various dual phase oral care composition may further comprise sodium benzoate from 0.1% to 0.9% by weight of the second part of the dual phase composition, xylitol from 5% to 10% by weight of the total oral care composition, sea salt (sodium chloride) (e.g., celtic sea salt) from 0.001% to 0.009% by weight of the second part of the dual phase composition, calcium lactate from 0.01% to 0.09% by weight of the second part of the dual phase composition, zinc gluconate from 0.001% to 0.05% by weight of the second part of the dual phase composition, stevia from 0.01% to 0.1% by weight of the second part of the dual phase composition, sodium bicarbonate from 0.01% to 5% by weight of the second part of the dual phase composition, aloe vera from 0.001% to 0.05% by weight of the second part of the dual phase composition, sorbitol 70% 8% to 15% by weight of the second part of the dual phase composition, glycerin from 12% to 18% by weight of the second part of the dual phase composition, PEG-40 hydrogenated castor oil from 0.3% to 1% by weight of the second part of the dual phase composition, papain from 0.1% to 0.5% by weight of the second part of the dual phase composition, sodium lauroyl sarcosinate from 0.001% to 0.01% by weight of the second part of the dual phase composition, clove oil from 0.01% to 0.1% by weight of the second part of the dual phase composition, spearmint oil from 0.001% to 0.1% by weight of the second part of the dual phase composition, tea tree oil from 0.001% to 0.1% by weight of the second part of the dual phase composition, peppermint oil from 0.01% to 0.1% by weight of the second part of the dual phase composition, cardamom oil from 0.01% to 0.1% by weight of the second part of the dual phase composition, oregano oil from 0.001% to 0.1% by weight of the second part of the dual phase composition, fennel oil from 0.001% to 0.1% by weight of the second part of the dual phase composition, jasmine oil from 0.0001% to 0.1% by weight of the second part of the dual phase composition, vitamin E from 0.01% to 0.1% by weight of the second part of the dual phase composition, vitamin D from 0.0001% to 0.1% by weight of the second part of the dual phase composition, vitamin K2 from 0.001% to 0.1% by weight of the second part of the dual phase composition, catalase from 0.01% to 0.1% by weight of the second part of the dual phase composition, and menthol from 0.0001% to 0.1% by weight of the second part of the dual phase composition.

In various embodiments, the first part of various dual phase oral care composition may comprise water from about 97% to about 98% by weight of the total first part of the dual phase oral care composition. The first part of various dual phase oral care composition may further comprise sodium benzoate from 0.1% to 0.5% by weight of the total oral care composition, citric acid from 0.001% to 0.005% by weight of the total oral care composition, hydrogen peroxide from 1.2% to 2% by weight of the total oral care composition.

In various embodiments, the second part of various dual phase oral care composition may comprise water from about 62% to about 65% by weight of the total second part of the dual phase oral care composition. The second part of various dual phase oral care composition may further comprise sodium benzoate from 0.4% to 0.6% by weight of the second part of the dual phase composition, xylitol from 9% to 11% by weight of the total oral care composition, sea salt (sodium chloride) (e.g., celtic sea salt) from 0.001% to 0.003% by weight of the second part of the dual phase composition, calcium lactate from 0.01% to 0.03% by weight of the second part of the dual phase composition, zinc gluconate from 0.01% to 0.02% by weight of the second part of the dual phase composition, stevia from 0.01% to 0.7% by weight of the second part of the dual phase composition, sodium bicarbonate from 0.01% to 0.02% by weight of the second part of the dual phase composition, aloe vera from 0.001% to 0.003% by weight of the second part of the dual phase composition, sorbitol 70% 9% to 11% by weight of the second part of the dual phase composition, glycerin from 14% to 15% by weight of the second part of the dual phase composition, PEG-40 hydrogenated castor oil from 0.3% to 0.5% by weight of the second part of the dual phase composition, papain from 0.1% to 0.3% by weight of the second part of the dual phase composition, sodium lauroyl sarcosinate from 0.001% to 0.003% by weight of the second part of the dual phase composition, clove oil from 0.01% to 0.02% by weight of the second part of the dual phase composition, spearmint oil from 0.005% to 0.009% by weight of the second part of the dual phase composition, tea tree oil from 0.001% to 0.009% by weight of the second part of the dual phase composition, peppermint oil from 0.07% to 0.09% by weight of the second part of the dual phase composition, cardamom oil from 0.01% to 0.03% by weight of the second part of the dual phase composition, oregano oil from 0.001% to 0.005% by weight of the second part of the dual phase composition, fennel oil from 0.001% to 0.009% by weight of the second part of the dual phase composition, jasmine oil from 0.0001% to 0.0009% by weight of the second part of the dual phase composition, vitamin E from 0.01% to 0.03% by weight of the second part of the dual phase composition, vitamin D from 0.0001% to 0.0005% by weight of the second part of the dual phase composition, vitamin K2 from 0.000008% to 0.00005% by weight of the second part of the dual phase composition, catalase from 0.03% to 0.07% by weight of the second part of the dual phase composition, and menthol from 0.0001% to 0.0009% by weight of the second part of the dual phase composition.

An exemplary embodiment of a first part of the dual phase composition is given in percentage by weight of the first part of the dual phase composition.

TABLE 1

| INGREDIENT | Range % (w/w) |
|---|---|
| Water | 90-100 |
| Sodium Benzoate | 0.1-0.5 |
| Citric Acid | 0.1-1.0 |
| Hydrogen Peroxide | 0.1-6.0 |

An exemplary embodiment of a second part of the dual phase composition is given in percentage by weight of the second part of the dual phase composition.

TABLE 2

| INGREDIENT | Range % (w/w) |
|---|---|
| Water | 50-100 |
| Sodium Benzoate | 0.0-0.5 |
| Xylitol | 0.1-20.0 |
| Celtic Sea Salt | 0.0001-2.000 |
| Calcium Lactate | 0.001-1.0 |
| Zinc Gluconate | 0.0001-1.0 |
| Stevia | 0.001-0.5 |
| Sodium Bicarbonate | 0.001-1.0 |
| Aloe Vera | 0.001-1.0 |
| Sorbitol 70% | 0.1-20.0 |
| Glycerin | 0.1-20.0 |
| PEG-40 Hydrogenated Castor Oil | 0.1-2.0 |
| Papain | 0.001-1 |
| Sodium Lauroyl Sarcosinate | 0.001-5.0 |
| Spearmint Oil | 0.001-1.0 |
| Clove Oil | 0.001-1.0 |
| Tea-Tree Oil | 0.001-1.0 |
| Peppermint Oil | 0.001-1.0 |
| Cardamom Oil | 0.001-1.0 |
| Oregano Oil | 0.001-1.0 |
| Fennel Oil | 0.001-1.0 |
| Jasmine Oil | 0.0001-1.0 |
| Vitamin E | 0.001-1.0 |
| Vitamin D | 0.001-1.0 |
| Catalase | 0.01-1.0 |
| Vitamin K2 | 0.00001-1.0 |
| Menthol | 0.0001-1.0 |

An exemplary embodiment of a first part of the dual phase composition is given in percentage by weight of the first part of the dual phase composition.

TABLE 3

| INGREDIENT | Range % (w/w) |
|---|---|
| Water | 96.0-99.0 |
| Sodium Benzoate | 0.3-0.6 |
| Citric Acid | 0.0009-0.002 |
| Hydrogen Peroxide | 1.0-2.0 |

An exemplary embodiment of a second part of the dual phase composition is given in percentage by weight of the second part of the dual phase composition.

TABLE 4

| INGREDIENT | Range % (w/w) |
|---|---|
| Water | 63.0-64.0 |
| Sodium Benzoate | 0.4-0.6 |
| Xylitol | 9.0-11.0 |
| Celtic Sea Salt | 0.0009-0.002 |
| Calcium Lactate | 0.009-0.002 |
| Zinc Gluconate | 0.01-0.02 |
| Stevia | 0.03-0.05 |
| Sodium Bicarbonate | 0.009-0.02 |
| Aloe Vera | 0.0009-0.002 |
| Sorbitol 70% | 9.0-11.0 |
| Glycerin | 14.8-15.4 |
| PEG-40 Hydrogenated Castor Oil | 0.35-0.5 |
| Papain | 0.1-0.3 |
| Sodium Lauroyl Sarcosinate | 0.0009-0.002 |
| Spearmint Oil | 0.005-0.01 |
| Clove Oil | 0.01-0.03 |
| Tea-Tree Oil | 0.003-0.009 |
| Peppermint Oil | 0.05-0.09 |
| Cardamom Oil | 0.01-0.03 |
| Oregano Oil | 0.001-0.009 |
| Fennel Oil | 0.001-0.009 |
| Jasmine Oil | 0.001-0.009 |
| Vitamin E | 0.007-0.03 |
| Vitamin D | 0.00009-0.0002 |
| Catalase | 0.03-0.07 |
| Vitamin K2 | 0.000008-0.00005 |
| Menthol | 0.0001-0.0006 |

### Packaging

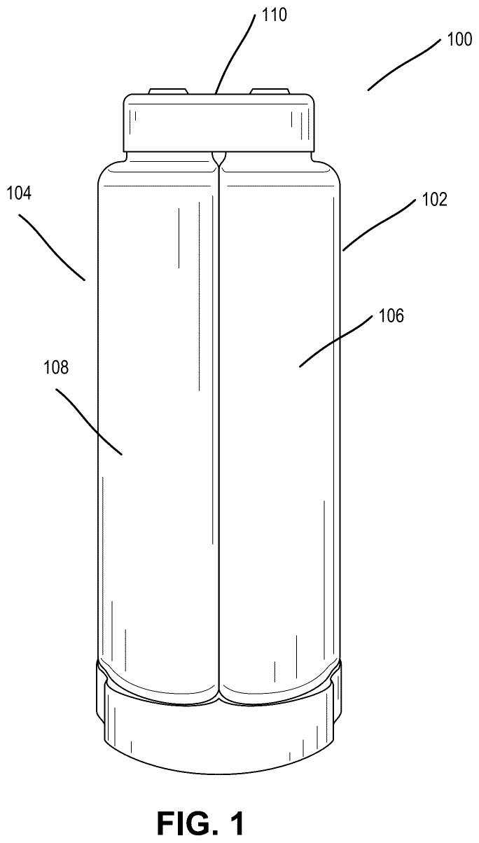

In various embodiments, the dual phase oral care composition is packaged in package 100, with reference to FIG. 1. Package 100 comprises vessel 102 and vessel 104. Vessel 102 and Vessel 104 define separate voids therein for containing first part 106 and second part 108. In this manner, first part 106 and second part 108 are held physically separate in package 100. Vessel 102 and vessel 104 are in fluid communication with spout 110. Upon decantation, first part 106 and second part 108 pass through spout 110 and come into physical contact. In this manner, first part and second part react as described herein, activating the dual phase oral care composition for use.

### Manufacture

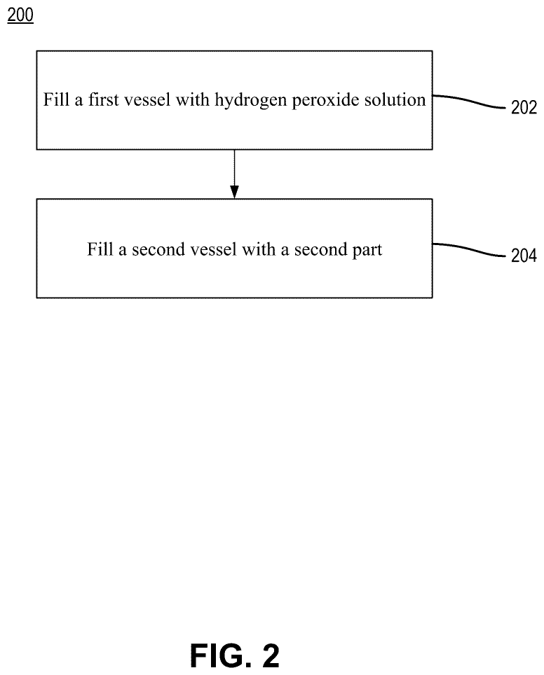

With reference to FIG. 2, a method of manufacture 200 is illustrated. A first vessel is filled 202 with a hydrogen peroxide solution. A second vessel is filled 204 with a second part as disclosed herein. The first vessel and second vessel may comprise vessel 102 and vessel 104 as described herein and in FIG. 1. In this manner, the dual phase oral care composition is prepare in package 100 for sale.

The detailed description shows embodiments by way of illustration, including the best mode. While these embodiments are described in sufficient detail to enable those skilled in the art to practice the principles of the present disclosure, it should be understood that other embodiments may be realized and that chemical changes may be made without departing from the spirit and scope of principles of the present disclosure. Thus, the detailed description herein is presented for purposes of illustration only and not of limitation. With regard to procedures, methods, techniques, and workflows that are in accordance with some embodiments, some operations in the procedures, methods, techniques, and workflows disclosed herein may be combined and/or the order of some operations may be changed. For example, the steps recited in any of the method descriptions may be executed in any suitable order and are not limited to the order presented.

In the above description, all cited references are incorporated herein by reference in their entireties. The citing of any reference is not an admission that such a reference is relevant prior art; rather, citations are to reference the novelty of the invention and discoveries described herein relative to known scientific literature, practices and prior art. In the description of the present disclosure, all ratios are weight ratios unless specifically stated otherwise. Unless otherwise indicated or evident from context, preferences indicated above and herein apply to the entirety of the embodiments discussed herein.

In describing the present disclosure, the following terminology will be used: The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” means quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc.

This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

The scope should be determined by the appended claims and their legal equivalents, rather than by the examples given above. For example, the operations recited in any method claims may be executed in any order and are not limited to the order presented in the claims. Moreover, no element is essential unless specifically described herein as “critical” or “essential.”

Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

1. An aqueous oral care composition for oral care comprising:
from about 0.001% to about 1% by weight sodium bicarbonate;
from about 8% to about 15% by weight xylitol;
from about 10% to about 17% by weight sorbitol 70%;
from about 12% to about 17% by glycerin; and
from about 60% to about 70% by weight water.

2. The aqueous oral care composition for oral care of claim 1, further comprising sodium benzoate from 0.1% to 0.0.5% by weight of the aqueous oral care composition.

3. The aqueous oral care composition for oral care of claim 2, further comprising stevia from 0.01% to 0.1% by weight of the aqueous oral care composition.

4. The aqueous oral care composition for oral care of claim 3, further comprising PEG-40 hydrogenated castor oil from 0.1% to 2% by weight of the aqueous oral care composition.

5. The aqueous oral care composition for oral care of claim 4, further comprising calcium lactate from 0.001% to 0.1% by weight of the aqueous oral care composition.

6. The aqueous oral care composition for oral care of claim 5, further comprising aloe vera from 0.001% to 0.0.1% by weight of the aqueous oral care composition.

7. The aqueous oral care composition for oral care of claim 6, further comprising zinc gluconate from 0.001% to 0.1% by weight of the aqueous oral care composition.

8. The aqueous oral care composition for oral care of claim 7, further comprising sodium lauroyl sarcosinate from 0.0001% to 0.0005% by weight of the aqueous oral care composition.

9. The aqueous oral care composition for oral care of claim 8, further comprising papain from 0.1% to 0.4% by weight of the aqueous oral care composition.

10. The aqueous oral care composition for oral care of claim 9, further comprising peppermint oil from 0.01% to 0.1% by weight of the aqueous oral care composition.

11. An oral care composition system for oral care comprising:
a container comprising a first vessel and a second vessel, the first vessel at least partially containing a hydrogen peroxide solution and the second vessel at least partially containing an activation composition,
wherein the hydrogen peroxide solution comprises:
from 1% to 3% hydrogen peroxide by weight of the hydrogen peroxide solution,
from 0.001% to 0.5% sodium benzoate by weight of the hydrogen peroxide solution,
and wherein the activation composition comprises:
from about 0.001% to about 1% by weight sodium bicarbonate;
from about 8% to about 15% by weight xylitol;
from about 10% to about 17% by weight sorbitol 70%;
from about 12% to about 17% by glycerin; and
from about 60% to about 70% by weight water.

12. The oral care composition system for oral care of claim 11, wherein the activation composition further comprises sodium benzoate from 0.1% to 0.5% by weight of the aqueous oral care composition.

13. The oral care composition system for oral care of claim 12, wherein the activation composition further comprises stevia from 0.01% to 0.1% by weight of the aqueous oral care composition.

14. The oral care composition system for oral care of claim 13, wherein the activation composition further comprises PEG-40 hydrogenated castor oil from 0.1% to 2% by weight of the activation composition.

15. The oral care composition system for oral care of claim 14, wherein the activation composition further comprises calcium lactate from 0.001% to 0.1% by weight of the activation composition.

16. The oral care composition system for oral care of claim 15, wherein the activation composition further comprises aloe vera from 0.001% to 0.005% by weight of the activation composition.

17. The oral care composition system for oral care of claim 16, wherein the activation composition further comprises zinc gluconate from 0.001% to 0.1% by weight of the activation composition.

18. The oral care composition system for oral care of claim 17, wherein the activation composition further comprises sodium lauroyl sarcosinate from 0.0001% to 0.0005% by weight of the activation composition.

19. The oral care composition system for oral care of claim 18, wherein the activation composition further comprises papain from 0.1% to 0.4% by weight of the activation composition.

20. The oral care composition system for oral care of claim 19, wherein the activation composition further comprises peppermint oil from 0.01% to 0.1% by weight of the activation composition.
